(19) United States
(12) Patent Application Publication
(10) Pub. No.: US 2024/0325711 A1
(43) Pub. Date: Oct. 3, 2024

(54) Tube Joining Device With Modular Accessories And Protocol Assurance

(71) Applicant: Terumo BCT, Inc., Lakewood, CO (US)

(72) Inventors: Shinu NAIR, Kerela (IN); Jeremy KOLENBRANDER, Brighton, CO (US); Jesse JANZEN, Golden, CO (US); Pierre R. EMERIC, Greenwood Village, CO (US); James R. LADTKOW, Broomfield, CO (US); Pierre PAYARD, Machelen (BE); Peter BAERTS, Waanrode (BE)

(21) Appl. No.: 18/615,750

(22) Filed: Mar. 25, 2024

Related U.S. Application Data
(60) Provisional application No. 63/455,748, filed on Mar. 30, 2023.

(51) Int. Cl. A61M 39/10; G06K 7/10; G06K 7/14

(52) U.S. Cl. CPC A61M 39/10; G06K 7/10297; G06K 7/10366; G06K 7/1413; G06K 7/1417

(57) ABSTRACT

A method of initiating a tube joining process includes receiving, with a tube joining device, data associated with a cartridge. The method further includes determining whether the cartridge is authentic based on the data. The method further includes, in response to a determination that the cartridge is authentic, initiating the tube joining process. A method of verifying media combination protocol includes verifying a first media bag. The verifying includes receiving, with a tube joining device, first data associated with the first media bag. The receiving includes concurrently scanning, with a scanner, a first plurality of items. The method further includes mapping the first plurality of items to a first plurality of predetermined fields. The method further includes, after the mapping, determining whether a first predetermined condition is met.

### CROSS-REFERENCE TO RELATED APPLICATION

This application claims the benefit of U.S. Provisional Application No. 63/455,748 filed on Mar. 30, 2023. The entire disclosure of the above application is incorporated herein by reference.

### FIELD

The present disclosure relates to a tube joining device with modular accessories and protocol assurance.

### BACKGROUND

This section provides background information related to the present disclosure which is not necessarily prior art.

Media bags, such as blood bags, may be used to collect and store blood or blood components obtained from donated blood. A blood bag generally includes a flexible plastic pouch connected to one end of a flexible tube of a predetermined length. When the blood is not being accessed, such as in storage, the blood bag is sealed by a weld at the free end of the flexible tube. When the blood needs to be accessed, the free end of the flexible tube is welded to another tube or bag in a sterile manner and/or accessed via a spike port on the bag. For example, the blood may be used in a blood transfusion system, a dialysis system, and the like.

Often, the blood is accessed for further processing and further storage. For example, the blood, stored in a sealed bag, may be separated into different components (typically blood plasma, red blood cells and white blood cells) by centrifugal separation. An individual blood component may be drawn from the blood bag and transferred to another blood bag for storage or further use. Blood bags containing different blood components may thus be produced, packaged and supplied to hospitals and clinics. When a blood component is needed, the flexible tube of the blood bag containing the blood component may be cut and connected to a piece of medical equipment, as above.

### SUMMARY

This section provides a general summary of the disclosure, and is not a comprehensive disclosure of its full scope or all of its features.

At least one example embodiment relates to a method of initiating a tube joining process. The method includes receiving, with a tube joining device, data associated with a cartridge. The method further includes determining whether the cartridge is authentic based on the data. The method further includes, in response to a determination that the cartridge is authentic, initiating the tube joining process.

In at least one example embodiment, the determining includes comparing the data to a predetermined list of data. The determining further includes determining that the cartridge is authentic if the data is included in the predetermined list of data.

In at least one example embodiment, the receiving includes scanning, with a scanner, an item.

In at least one example embodiment, the item is a one-dimensional barcode, a two-dimensional barcode, a radiofrequency identification, a near-field communication tag, or any combination thereof.

In at least one example embodiment, the method further includes, in response to a determination that the cartridge is not authentic, generating an alert.

In at least one example embodiment, the generating includes generating a visual alert, an audible alert, a tactile alert, or any combination thereof.

At least one example embodiment relates to a method of verifying media combination protocol. The method includes verifying a first media bag. The verifying includes receiving, with a tube joining device, first data associated with the first media bag. The receiving includes concurrently scanning, with a scanner, a first plurality of items. The method further includes mapping the first plurality of items to a first plurality of predetermined fields. The method further includes, after the mapping, determining whether a first predetermined condition is met.

In at least one example embodiment, the determining includes determining whether a respective first predetermined condition is met for each of the first plurality of predetermined fields.

In at least one example embodiment, the method further includes verifying a second media bag. The verifying the second media bag includes receiving, with the tube joining device, second data associated with the second media bag. The receiving includes concurrently scanning, with a scanner, a second plurality of items. The method further includes mapping the second plurality of items to a second plurality of predetermined fields. The method further includes after the mapping, determining whether a second predetermined condition is met.

In at least one example embodiment, the determining whether the first predetermined condition is met is based on the first data and data stored in a memory of the tube joining device.

In at least one example embodiment, the determining whether the second predetermined condition is met is based on the first data, the second data, and the data stored in the memory of the tube joining device.

In at least one example embodiment, the method further includes performing, with the tube joining device, a tube joining process based on a determination that the first predetermined condition and the second predetermined condition are met.

In at least one example embodiment, the method further includes storing, in a memory of the tube joining device, the first data associated with the first media bag.

In at least one example embodiment, wherein the first plurality of items includes a plurality of one-dimensional barcodes, a plurality of two-dimensional barcodes, a plurality of radiofrequency identifications, or a plurality of near-field communication tags.

In at least one example embodiment, the determining is based on the first plurality of items and data stored in a memory of the tube joining device.

In at least one example embodiment, the method further includes based on a determination that the first predetermined condition is not met, generating an alert.

In at least one example embodiment, the first data associated with the first media bag includes media bag identification, storage solution, sex of donor, viral status of media, blood type, quantity of media units, unit identification, process step, or any combination thereof.

At least one example embodiment relates to a method of performing a tube joining process. The method includes applying a current to a wafer. The method further includes determining a temperature of the wafer during the applying. The method further includes determining whether the temperature is within a predetermined temperature range. The method further includes, based on a determination that the temperature is within the predetermined temperature range, performing, with a tube joining device, a tube joining process.

In at least one example embodiment, the determining the temperature includes detecting an electrical resistance of the wafer. The determining the temperature further includes calculating the temperature based on the electrical resistance.

In at least one example embodiment, the method further includes, based on a determination that the temperature is outside of the predetermined temperature range, generating an alert.

In at least one example embodiment, the method further includes, based on a determination that the temperature is outside of the predetermined temperature range, replacing the wafer.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a tube joining device according to at least one example embodiment.

FIG. 2A is a top perspective view of a cartridge of the tube joining device of FIG. 1 according to at least one example embodiment.

FIG. 2B is a bottom perspective view of the cartridge of FIG. 2A.

FIG. 3 is a perspective view of a wafer of the cartridge of FIGS. 2A-2B according to at least one example embodiment.

FIG. 4 is a partial perspective sectional view the tube joining device of FIG. 1 taken along line 4-4 of FIG. 1 according to at least one example embodiment.

FIG. 5 is a partial sectional view of the tube joining device of FIG. 1 taken along line 5-5 with a wafer in a pre-joining position according to at least one example embodiment.

FIG. 6 is an exploded perspective view of the tube joining device of FIG. 1 according to at least one example embodiment.

FIG. 7 is a partial perspective view of a base of the tube joining device of FIG. 1 according to at least one example embodiment.

FIG. 8 is a perspective view of a discrete elongated connector of the tube joining device of FIG. 1 according to at least one example embodiment.

FIG. 9 is a perspective view of an elongated blank component for use with the tube joining device of FIG. 1 according to at least one example embodiment.

FIG. 10 is a partial perspective view of the tube joining device of FIG. 1 according to at least one example embodiment.

FIG. 11 is a partial schematic sectional view of the tube joining device of FIG. 10 taken at line 11-11 according to at least one example embodiment.

FIG. 12 is a perspective view of a scanner subassembly of the tube joining device of FIG. 1 according to at least one example embodiment.

FIG. 13 is a schematic elevation view of a base of the tube joining device of FIG. 1 according to at least one example embodiment.

FIG. 14 is a schematic elevation view of a tube joining system including the tube joining device of FIG. 1 according to at least one example embodiment.

FIG. 15 is a flowchart depicting a method tube joining including cartridge authenticity verification according to at least one example embodiment.

FIG. 16 is a flowchart depicting a method of verifying media combination protocol according to at least one example embodiment.

FIG. 17 is a perspective view of the tube joining device of FIG. 1 having a media bag on a top wall according to at least one example embodiment.

FIG. 18 is a flowchart depicting a method of performing a tube joining process with wafer verification according to at least one example embodiment.

Corresponding reference numerals indicate corresponding parts throughout the several views of the drawings.

### DETAILED DESCRIPTION

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. As used herein, the term “and/or” includes any and all combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, component, region, layer, or section discussed below could be termed a second element, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below”, or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” specify the presence of stated features, integers, steps, operations, and/or elements, but do not preclude the presence or addition of one or more other features, integers, steps, operations, elements, and/or groups thereof.

When the terms “about” or “substantially” are used in this specification in connection with a numerical value, it is intended that the associated numerical value includes a manufacturing or operational tolerance (e.g., ±10%) around the stated numerical value. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

Hardware may be implemented using processing or control circuitry such as, but not limited to, one or more processors, one or more Central Processing Units (CPUs), one or more microcontrollers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUS), one or more microprocessors, one or more Application Specific Integrated Circuits (ASICs), or any other device or devices capable of responding to and executing instructions in a defined manner.

One or more example embodiments may be described herein, in at least some instances, as being performed by a tube joining device including at least one processor and a memory storing computer-executable instructions, wherein the at least one processor is configured to execute the computer-readable instructions to cause the tube joining device to perform operations of one or more example embodiments. Additionally, the processor, memory and example algorithms, encoded as computer program code, may serve as means for providing or causing performance of operations discussed herein.

As discussed above, media, such as blood, may be stored in a bag with an attached tube that is sealed, such as with a weld. To access the media in the bag, the weld in the free end of the flexible tube may be cut away and connected to another tube. The cutting and joining are generally performed in sterile conditions for increased medical safety. A new connection may be formed by joining together the flexible tube of the blood bag to another flexible tube. The weld provides a leak-free connection and good mechanical strength. Because a length of flexible tube is lost every time it is cut, the original length of the flexible tube may be sufficient to allow it to be cut (and welded) several times.

Tube-joining devices are used for joining together two flexible tube sections. In general, two flexible tube sections to be joined are installed into the tube-joining device in parallel next to each other. The tube-joining device then cuts the flexible tube sections and melts the cut faces, such as using a heated blade. The tube-joining device then displaces the flexible tube sections on one side of the blade so that a cut face of one tube is aligned with a cut face of the other tube. Upon withdrawal of the blade, said cut face of one tube is allowed to contact said cut face of the other tube, thereby forming a welded joint. The flexible tube sections are then released from the device.

At least one example embodiment of the present disclosure relates to a tube joining device. The tube joining device may be configured to form a plurality of dovetail joints with subassemblies, structures, and/or other devices (e.g., additional tube joining devices). The dovetail joints may include ground, power, and/or data connections. Connection regions may be arranged such that coupling of subassemblies is modular. The tube joining device may be configured to perform protocol verification tasks prior to beginning a joining process. Protocol verification may include cartridge authenticity verification, media combination verification, and/or wafer quality verification.

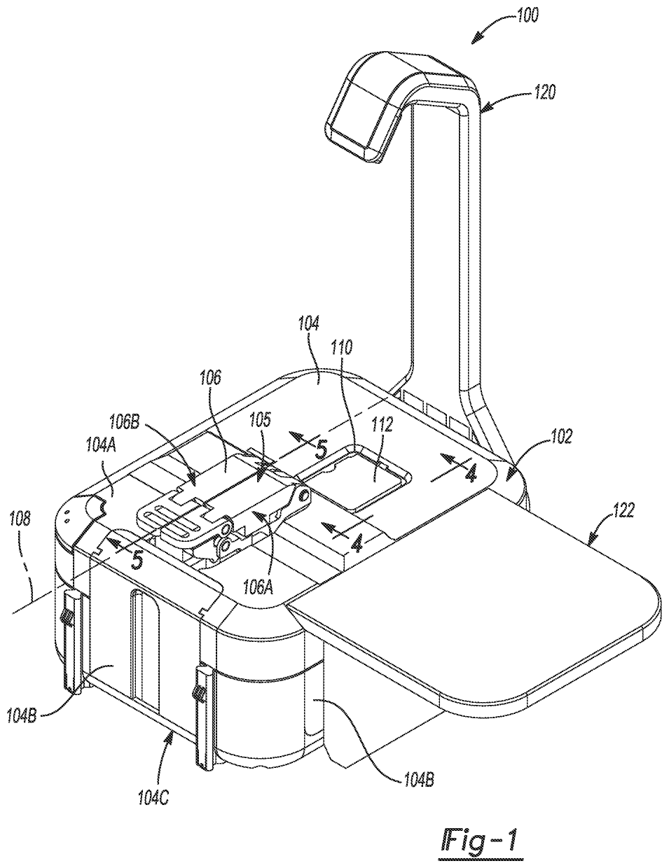

FIG. 1 is a perspective view of a tube joining device according to at least one example embodiment.

At least one example embodiment relates to a tube joining device or tube welding device 100, as shown in FIG. 1. The tube joining device 100 includes a base assembly 102 having a housing 104. The housing 104 may include a top wall 104A, a plurality of side walls 104B, and a bottom wall 104C. The tube joining device 100 may include a tube joining subassembly 105. The tube joining subassembly may be at least partially within the housing 104 and/or coupled to the housing 104 (either directly or indirectly).

In at least one example embodiment, the tube joining subassembly 105 includes a tubing clamp 106. The tubing clamp 106 may be configured to clamp and/or retain first and second tubes (not shown) that are to be joined or welded together. The tubing clamp 106 may be attached the housing 104. In at least the example embodiment shown, the tubing clamp 106 includes a first clamp portion 106A and a second clamp portion 106B. At least one of the first and second clamp portions 106A, 106B is configured to translate parallel to a clamp axis 108 with respect to the other of the first and second clamp portions 106A, 106B, such as during a joining process. In at least one example embodiment, each of the tubing clamp portions 106A, 106B includes a fixed bottom portion and a pivotable top portion configured to pivot away from the fixed bottom portion to receive a tube therebetween.

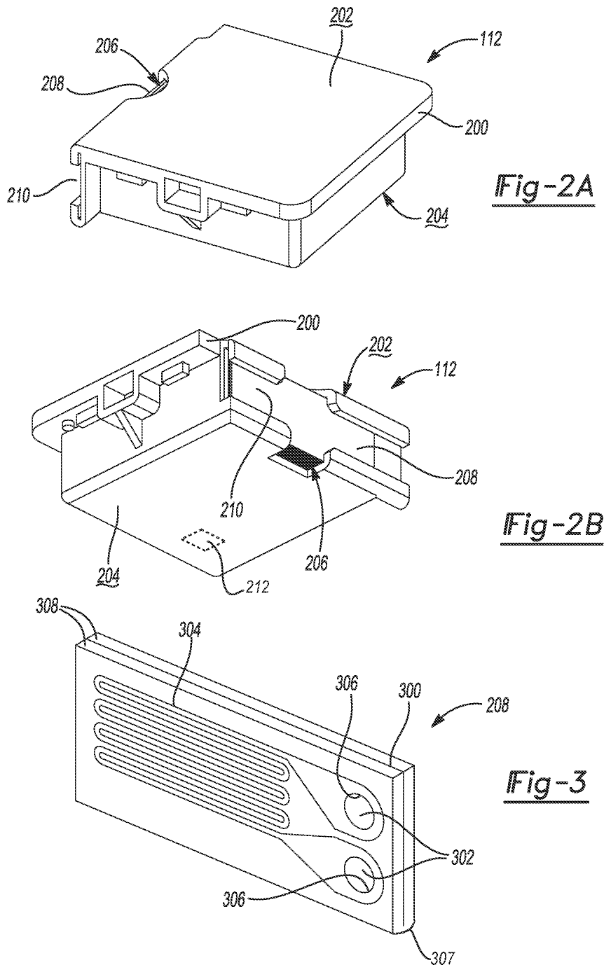

In at least one example embodiment, the housing 104 may define a cartridge receptacle 110. The cartridge receptacle 110 may be configured to at least partially receive a cartridge 112. The cartridge 112 may be recessed, flush, or protruding with respect to an outer surface of the housing 104. The cartridge 112 may contain a plurality of wafers (see, e.g., wafers 208 of FIG. 2B). The wafers may be configured to be heated, cut the first and second tubes, and/or melt and/or soften a portion of the first and/or second tube so they can be joined.

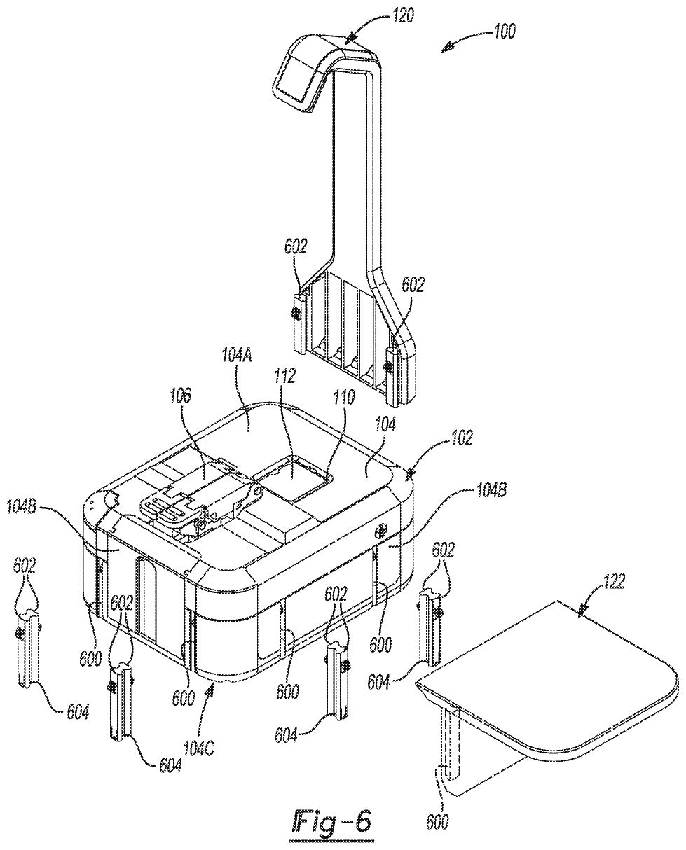

In at least one example embodiment, the tube joining device 100 includes a plurality of subassemblies, such as a scanner subassembly 120 and a table subassembly 122. In at least one other example embodiment, a tube joining device may include different or additional subassemblies. Other subassemblies may include a particle and/or smoke evacuator (e.g., vacuum) subassembly, a power supply subassembly, a weld opening subassembly, an accessory connection (e.g., USB accessory plug) subassembly, a handheld scanner subassembly (in addition to or as an alternative to the scanner subassembly 120), a light subassembly, a media bag hanger, a data entry device and/or subassembly, or any combination thereof. In at least one example embodiment, each of the subassemblies may be coupled to the base assembly 102 by one or more dovetail connections or joints, as will be described in greater detail below in the discussion accompanying FIGS. 6-14. In at least one example embodiment, a tube joining device may further include a radiofrequency identification (RFID) antenna (not shown).

FIG. 2A is a top perspective view of a cartridge of the tube joining device of FIG. 1 according to at least one example embodiment. FIG. 2B is a bottom perspective view of the cartridge of FIG. 2A.

In at least one example embodiment, as shown in FIGS. 2A-2B, the cartridge 112 includes a cartridge housing 200. The cartridge housing 200 includes a top surface 202 and a bottom surface 204. The top surface 202 may be configured to be visible from an exterior of the tube joining device 100 (shown in FIG. 1). The bottom surface 204 may be configured to be in communication with an interior of the housing 104 (shown in FIG. 1) of the tube joining device 100.

The cartridge housing 200 at least partially defines an interior region 206. A plurality of wafers 208 is at least partially within the interior region 206. In at least one example embodiment, the plurality of wafers 208 includes a quantity of wafers 208 ranging from about 10 wafers to about 200 wafers (e.g., greater than or equal to about 10 wafers, greater than or equal to about 25 wafers, greater than or equal to about 50 wafers, greater than or equal to about 60 wafers, greater than or equal to about 70 wafers, greater than or equal to about 80 wafers, greater than or equal to about 90 wafers, greater than or equal to about 100 wafers, greater than or equal to about 125 wafers, greater than or equal to about 150 wafers, or greater than or equal to about 175 wafers; less than or equal to about 175 wafers, less than or equal to about 200 wafers, less than or equal to about 175 wafers, less than or equal to about 150 wafers, less than or equal to about 125 wafers, less than or equal to about 100 wafers, less than or equal to about 90 wafers, less than or equal to about 80 wafers, less than or equal to about 70 wafers, less than or equal to about 60 wafers, less than or equal to a bout 50 wafers, or less than or equal to about 25 wafers). In at least one example embodiment, the cartridge housing 200 further defines an opening or slot 210. At least a portion of the plurality of wafers 208 may be in communication with the opening 210.

In at least one example embodiment, the cartridge 112 may include a unique item or identifier 212. The unique identifier 212 may be a barcode (e.g., a one-dimensional barcode or a two-dimensional barcode), a number, a series of letters, an alpha-numeric code, a radiofrequency identification (RFID), a near-field communication (NFC) tag, an image, or any combination thereof. In at least one example embodiment, the unique identifier may be on the bottom surface 204 of the cartridge 112.

FIG. 3 is a perspective view of a wafer of the cartridge of FIGS. 2A-2B according to at least one example embodiment.

In at least one example embodiment, each of the wafers 208 includes a body 300. The body 300 defines one or more apertures, depressions, and/or protrusions, such as a pair of apertures 302, as shown. The wafer 208 further includes a resistive trace 304 (also referred to as a “circuit trace”) in thermal communication with the body 300. The resistive trace 304 may be in electrical communication with surfaces 306 that at least partially define the respective apertures 302.

In at least one example embodiment, the body 300 is thermally conductive. In at least one example embodiment, the body 300 is formed from or includes copper. In at least the example embodiment shown, the body 300 may be formed from a copper sheet. The copper sheet may be folded onto itself at a folded edge 307 such that the body 300 includes two layers 308. In the example embodiment shown, the folded edge 307 is along a long edge of the wafer 208. The folded edge 307 may the leading or cutting edge in a tube joining operation. In at least one other example embodiment, a fold may be on another edge of a wafer.

The resistive trace 304 may be on, within, and/or embedded in the body 300. In at least the example embodiment shown, the resistive trace 304 between the two layers 308 of the body 300. In at least one example embodiment, the resistive trace 304 may be printed onto a surface (e.g., an interior surface when the sheet is folded onto itself) of the body 300 (e.g., the copper sheet prior to being folded onto itself). A material of the resistive trace 304 may be selected to achieve a desired electrical impedance. In at least one example embodiment, the resistive trace 304 is formed from and/or includes silver, such as silver paste. In at least one other example embodiment, additionally or alternatively, the resistive trace 304 is formed from and/or includes graphite.

In at least one example embodiment, as shown, the resistive trace 304 is electrically conductive. The resistive trace 304 may be electrically isolated from the body 300. In at least one other example embodiment, a wafer body is itself electrically conductive and is free of a resistive trace. In this example, the wafer may be heated by passing current directly through the body of the wafer.

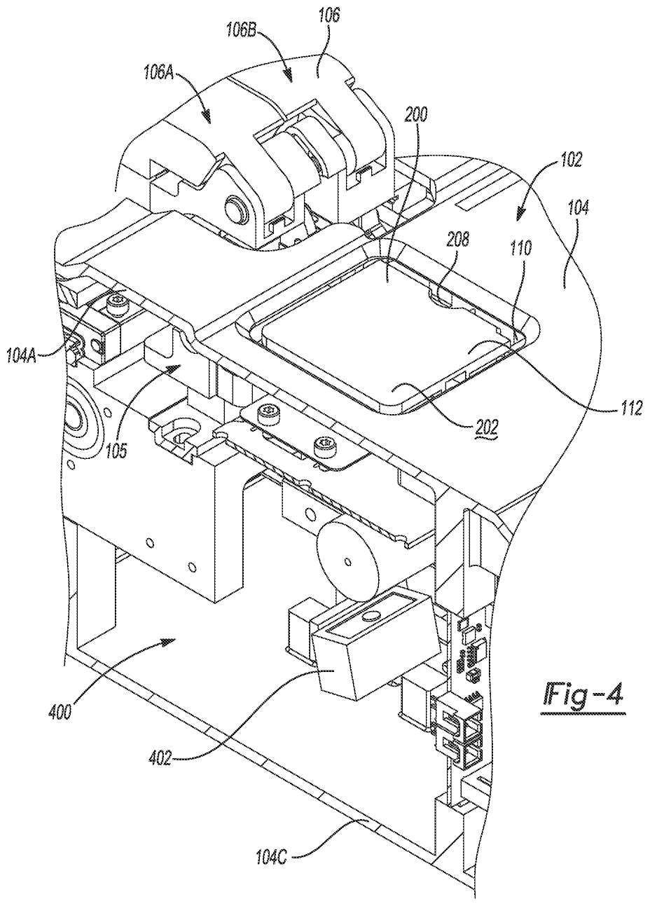

FIG. 4 is a partial perspective sectional view the tube joining device of FIG. 1 taken along line 4-4 of FIG. 1 according to at least one example embodiment.

In at least one example embodiment, as shown in FIG. 4, the housing 104 of the base assembly 102 at least partially defines an interior region 400. The tube joining subassembly 105 may be at least partially in the interior region 400. The bottom surface 204 of the cartridge 112 (shown in FIG. 2B) may be in communication with the interior region 400. In at least one example embodiment, the base assembly 102 further includes a scanner 402. The scanner 402 may be configured to read the unique identifier 212 (shown in FIG. 2B) on the cartridge 112. The scanner 402 may be a barcode scanner (e.g., for reading one-dimensional and/or two-dimensional barcodes), an RFID reader, an NFC reader, a camera, or any combination thereof.

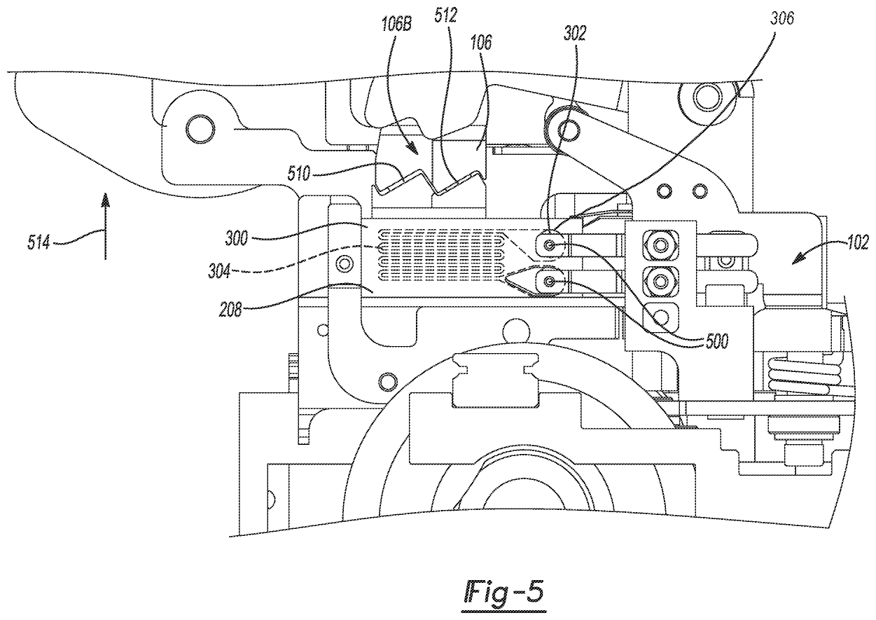

In at least one example embodiment, the tube joining device 100 is configured to individually and sequentially dispense the wafers 208 from the cartridge 112. The tube joining device 100 may include an arm (not shown) operatively coupled to a motor (not shown). The arm may engage wafer 208-1 at the opening 210 (shown in FIG. 2B) to move the wafer 208-1 from the cartridge 112 to a pre-joining or intermediate position away from the cartridge (shown in FIG. 5).

FIG. 5 is a partial sectional view of the tube joining device of FIG. 1 taken along line 5-5 with a wafer in a pre-joining position according to at least one example embodiment.

In at least one example embodiment, as shown in FIG. 5, the base assembly 102 includes a pair of electrical contacts 500. The electrical contacts 500 are at least partially in the apertures 302 of the wafer 208. The electrical contacts 500 are in electrical communication with the resistive trace 304 of the wafer 208, such as via the surfaces 306.

The clamp 106 defines a first channel 510 and a second channel 512. The first and second channels 510, 512 are configured to receive at least a portion of each of the first and second tubes to be welded to one another. The wafer 208 is configured to be moved in a first or upward direction 514 to cut the first and second tubes during a joining process.

FIG. 6 is an exploded perspective view of the tube joining device of FIG. 1 according to at least one example embodiment.

In at least one example embodiment, as shown in FIG. 6, each of the subassemblies (e.g., the scanner subassembly 120 and the table subassembly 122) is connected to the base assembly 102 by a dovetail joint or connection. In the example embodiment shown, each of the dovetail joints is an elongated dovetail joint. As will be described in greater detail below, an elongated component is at least partially in an elongated channel and engages a wall of the elongated channel to retain the respective subassembly coupled to the base assembly 102.

Each of the elongated dovetail joints includes one or more elongated channels 600 and one or more respective elongated protrusions 602. The channels 600 and the protrusions 602 may extend along respective elongated axes and define dovetail-shaped cross sections perpendicular to the longitudinal axes.

The channels 600 may be defined by the housing 104 of the base assembly 102 and/or the respective subassembly. The protrusions 602 may be present on the housing 104 of the base assembly 102, the respective subassembly (e.g., the scanner subassembly 120, the table subassembly 122), and/or a distinct elongated connector, component, or rod 604.

Portions of the elongated dovetail joints, such as the channels 600 and/or protrusions 602 may be present on the top, side, and/or bottom walls 104A, 104B, 104C of the housing 104 of the base assembly 102. As discussed above, the elongated dovetail joints may be used to couple subassemblies (e.g., the scanner subassembly 120, the table subassembly 122) to the base assembly 102. Additionally or alternatively, in at least one other example embodiment, a base assembly includes a portion of a dovetail joint on a bottom wall and another portion of the dovetail joint on a surface (e.g., a table, a cart) and the dovetail joint is used to couple the base assembly to the surface. Additionally or alternatively, as will be described in greater detail below, in at least one other example embodiment, a dovetail joint is used to couple a plurality of tube joining devices to one another. Additionally or alternatively, in at least one other example embodiment, a dovetail joint is used to couple a tube joining device to other equipment and/or a structure (e.g., a wall).

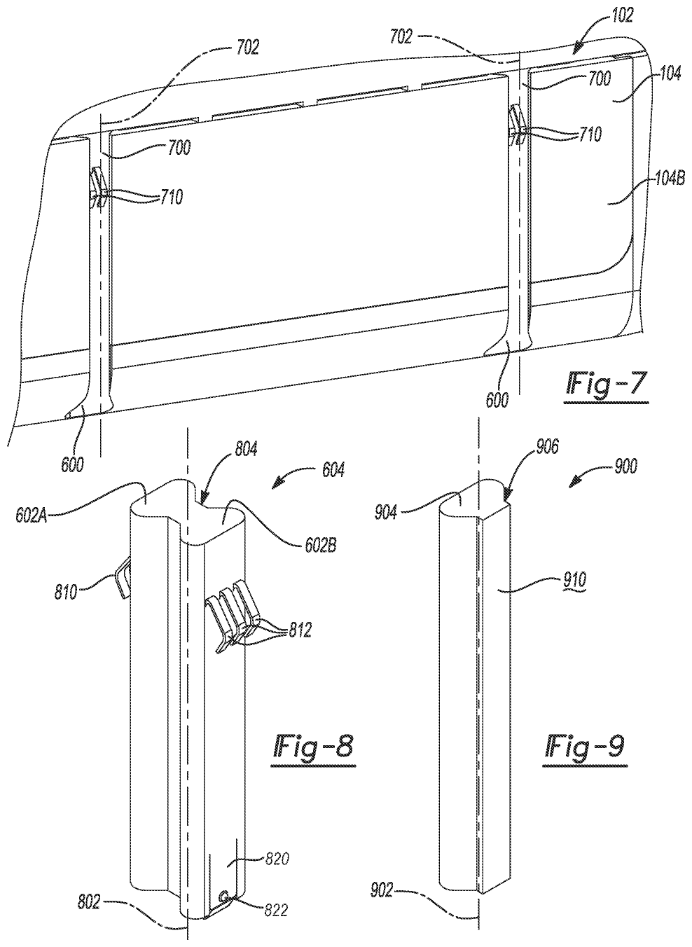

FIG. 7 is a partial perspective view of a base of the tube joining device of FIG. 1 according to at least one example embodiment.

In at least one example embodiment, as shown in FIG. 7, each channel 600 may be at least partially defined by a channel wall 700. The channel 600 may extend along a channel axis 702. The channel wall 700 may define a dovetail-shaped cross section perpendicular to a channel axis 702. However, a channel wall according to the present disclosure may define any other geometry capable of engaging an elongated protrusion.

In at least one example embodiment, one or more first contacts 710 are in communication with each of the channels 600. The first contacts 710 may within and/or otherwise accessible from the channels 600, such as protruding into the channels 600, as shown. The first contacts 710 may be electrically conductive and corrosion resistant. In at least one example embodiment, the first contacts 710 are formed from or include gold, silver, copper, bronze, brass, or any combination thereof. In at least one example embodiment, each of the first contacts 710 extends from the interior region 400 (shown in FIG. 4) of the housing 104 into the channel 600.

The first contacts 710 may be configured to form any desired connections with the base assembly 102. The first contacts 710 may each be in electrical communication with the base assembly 102 to form a ground connection, an electrical power connection, and/or an electrical data connection with the base assembly 102. In at least one example embodiment, the first contacts 710 may be electrically connected to a first printed circuit board assembly (PCBA), which may connected to a second PCBA via wires. The second PCBA may be a main PCBA for the tube welding device 100. In the example embodiment shown, each channel 600 is in communication with three first contacts 710; however, other channels may be free of first contacts, include a single first contact, include two first contacts, or include more than three first contacts. Each of the first contacts 710 may be electrically insulated from the other first contacts 710, such as by plastic.

As discussed above, channels 600 may be present on the base assembly 102, as shown, and/or on subassemblies. In at least one example embodiment, a subassembly may be coupled to the base assembly 102 via two elongated dovetail joints: a first joint between the base assembly 102 and a discrete elongated connector (see, e.g., discrete elongated connector 604 of FIG. 8) and a second joint between the discrete elongated connector and the subassembly.

FIG. 8 is a perspective view of a discrete elongated connector of the tube joining device of FIG. 1 according to at least one example embodiment.

In at least one example embodiment, as shown in FIG. 8, a discrete elongated connector or rod 604 extends along a connector axis 802. The discrete elongated connector 604 may include a central portion 804, a first elongated protrusion 602A, and a second elongated protrusion 602B. Each of the elongated protrusions 602A, 602B extends substantially parallel to the connector axis 802. Each of the elongated protrusions 602A, 602B may define a dovetail shape perpendicular to the connector axis 802. The elongated connector 604 may define a bowtie shape perpendicular to the connector axis 802. However, an elongated connector may define another other shape capable of being retained in an associated channel (e.g., a complementary shape and/or interlocking shape).

In at least one example embodiment, the first elongated protrusion 602A is configured to be at least partially in one of the channels 600 of the base assembly 102. The first elongated protrusion 602A may engage the channel wall 700 to couple the connector 604 to the base assembly 102. The first elongated protrusion 602A may have an interlocking relationship with the channel wall 700.

The first elongated protrusion 602A may include one or more second contacts 810. The second contacts 810 may be electrically conductive and corrosion resistant. In at least one example embodiment, the second contacts 810 are formed from or include gold, silver, copper, bronze, brass, or any combination thereof. The second contacts 810 are configured to be in electrical communication (e.g., indirect electrical communication, or direct electrical communication, as shown) with the first contacts 710 of the channel 600 (shown in FIG. 7). In the example embodiment shown, the first elongated protrusion 602A includes three second contacts 810; however, other embodiments may be free of second contacts, include a single second contact, include two second contacts, or include more than three second contacts. Each of the second contacts 810 may be electrically insulated from the other second contacts 810, such as by plastic.

In at least one example embodiment, the second elongated protrusion 602B is configured to be at least partially in a channel of a subassembly (e.g., the table subassembly 122 shown in FIG. 6). The second elongated protrusion 602B may include one or more third contacts 812. The third contacts 812 may be electrically conductive and corrosion resistant. In at least one example embodiment, the third contacts 812 are formed from or include gold, silver, copper, bronze, brass, or any combination thereof. The third contacts 812 are configured to be in electrical communication (e.g., direct electrical communication) with the fourth contacts of one of the subassemblies. The fourth contacts may be at least partially in a channel of the subassembly, similar or identical to the channel 600 and first contacts 710 of the base assembly 102 (shown in FIG. 7). In the example embodiment shown, the second elongated protrusion 602B includes three third contacts 812; however, other embodiments may be free of third contacts, include a single third contact, include two third contacts, or include more than three third contacts. Each of the third contacts 810 may be electrically insulated from the other third contacts 812, such as by plastic.

In at least one example embodiment, the second and third contacts 810, 812 are in respective electrical communication. The second and third contacts 810, 812 may be in respective contact in an interior of the central portion 804 of the elongated connector 604. Example schematic electrical connections are shown at 1100. The second and third contacts 810, 812 may be part of a single integral component that extends through the central portion 9804 of the elongated connector, or part of discrete, but electrically connected, components. In at least one example embodiment, an elongated connector rod may include a plurality of electrically conductive plates (e.g., three plates when there are three second contacts and three third contacts) separated by plastic, with each of the three plates including one of the second contacts and one of the third contacts. The second and third contacts 810 may be configured to cooperate with respective first contacts 710 to form connections or paths, such as a ground connection, an electrical power connection, or an electrical data connection.

In at least one example embodiment, one or both of the elongated protrusions 602A, 602B includes a retaining feature to retain the elongated connector 604 in the base assembly 102 (shown in FIG. 7) and/or a subassembly. In at least one example embodiment, the retaining feature includes a flexible tab 820 including a protrusion 822. In the relaxed position, as shown, the protrusion 822 may engage a portion of the base assembly 102 or subassembly. The flexible tab 820 may be flexed inward to disengage the protrusion 822 from the base assembly 102 or the subassembly so that the elongated connector 604 can be removed from the base assembly 102 or the subassembly. In at least one other example embodiment, a retaining feature includes a ball detent, a latch, a clasp, a screw, a quarter turn fastener, or any combination thereof.

FIG. 9 is a perspective view of an elongated blank component for use with the tube joining device of FIG. 1 according to at least one example embodiment.

In at least one example embodiment, as shown in FIG. 9, an elongated blank component 900 may extend along a blank axis 902. The elongated blank 900 may include an elongated protrusion 904 and a planar portion 906. The elongated protrusion 904 may be similar to the elongated protrusions 602A, 602B of the elongated connector 604 of FIG. 8. In at least the example embodiment shown, the elongated blank 900 may be free of contacts.

In at least one example embodiment, the elongated blank 900 is configured to be at least partially in a channel 600 (shown in FIG. 7) of the base assembly 102 (shown in FIG. 7) or a subassembly. In at least one example embodiment, the elongated blank 900 is configured to be fully within the channel 600 such that a surface 910 of the planar portion 906 is coplanar or flush with an outer surface of the housing 104 (shown in FIG. 7). Accordingly, use of the blank 900 may improve aesthetics and/or safety (e.g., by reducing or eliminating pinch points and/or reducing or preventing exposure of electrical contact points) of the tube joining device 100 (shown in FIG. 1). In at least one example embodiment, the elongated blank component 900 further includes a retaining feature to retain the elongated blank 900 in the base assembly 102 (see, e.g., flexible tab 820 in FIG. 8).

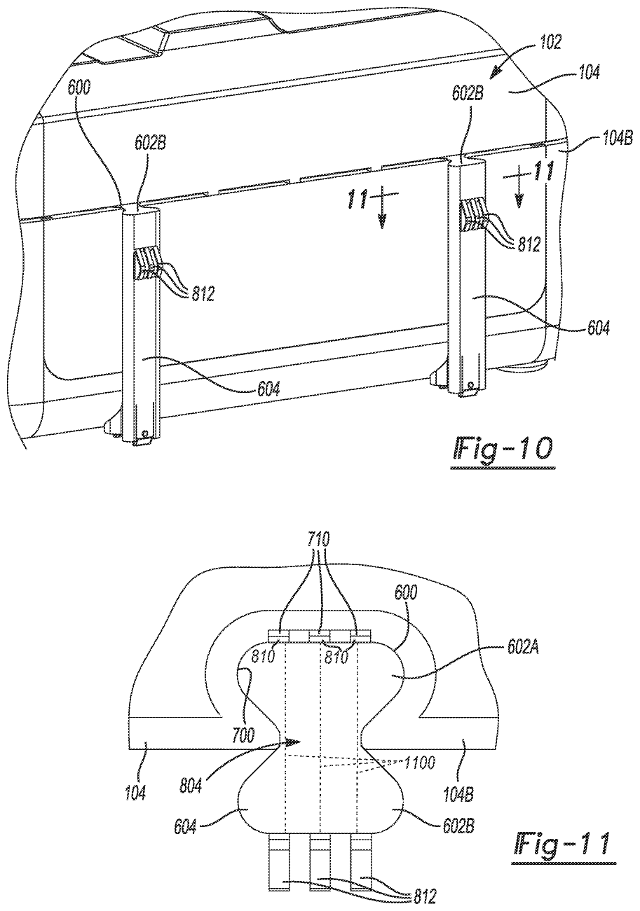

FIG. 10 is a partial perspective view of the tube joining device of FIG. 1 according to at least one example embodiment. FIG. 11 is a partial sectional schematic view of the tube joining device of FIG. 10 taken at line 11-11 according to at least one example embodiment.

In at least one example embodiment, as shown in FIGS. 10-11, the elongated connectors 604 are configured to be at least partially in the channels 600. The first elongated protrusion 602A of the elongated connector 604 may be in the channel 600. When the elongated connector 604 is in the channel 600, the first contacts 710 are in electrical communication with the second contacts 810. The first contacts 710 may be in direct physical contact with the second contacts 810. In at least one example embodiment, the first and/or second contacts 710, 810 may deform, such as by engaging one another to compress, as springs. When the elongated connector 604 is removed from the channel 600, the first and second contacts 710, 810 may return to their respective original shapes.

In at least one example embodiment, the elongated connector 604 may be configured to be at least partially in a channel of a subassembly (e.g., the scanner subassembly 120, the table subassembly 122). The second elongated protrusion 602B of the elongated connector 604 may be in the subassembly channel. The third contacts 812 may engage fourth contacts (not shown) of the subassembly. When the elongated connector 604 is in the subassembly channel, the third contacts 812 are in electrical communication with the fourth contacts. The third contacts 812 may be in direct physical contact with the fourth contacts. In at least one example embodiment, the third contacts 812 and/or fourth contacts may deform, such as by engaging one another to compress, as springs. When the elongated connector 604 is removed from the subassembly channel, the third contacts 812 and fourth contacts may return to their respective original shapes.

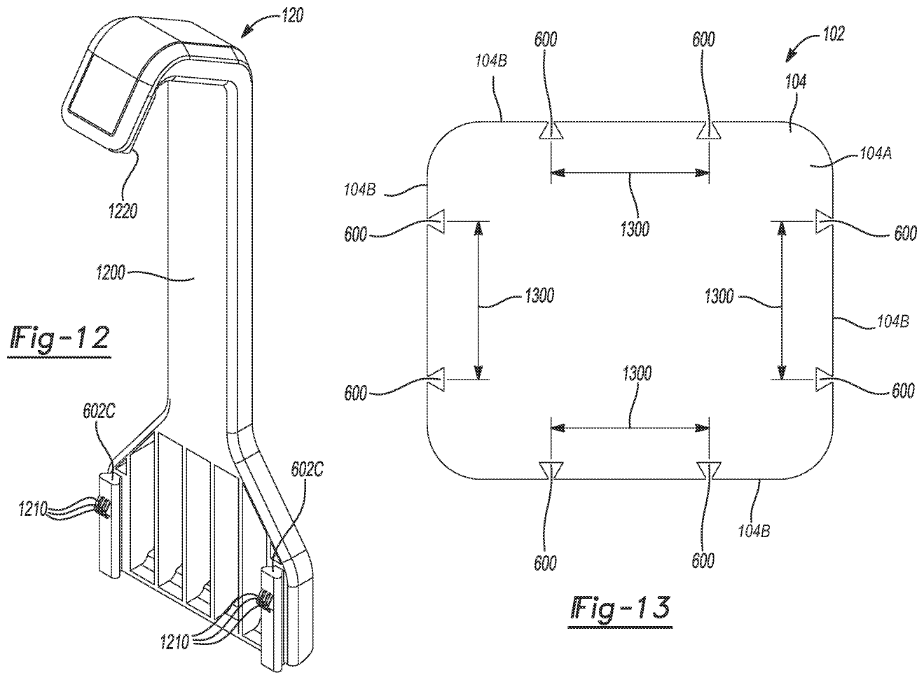

FIG. 12 is a perspective view of a scanner subassembly of the tube joining device of FIG. 1 according to at least one example embodiment.

In at least one example embodiment, as shown in FIG. 12, one or more elongated protrusions 602C (also referred to as “integral elongated protrusions”) may be integral with a subassembly, such as the scanner subassembly 120. The elongated protrusion 602 may be integrally formed with a body 1200 of the subassembly, such as the scanner subassembly 120 and/or permanently attached to the scanner subassembly 120.

In at least the example embodiment shown, the integral elongated protrusions 602C include one or more fifth contacts 1210. The fifth contacts 1210 may be electrically conductive and corrosion resistant. In at least one example embodiment, the fifth contacts 1210 are formed from or include gold, silver, copper, bronze, brass, or any combination thereof. The fifth contacts 1210 are configured to be in electrical communication (e.g., direct electrical communication) with the first contacts 710 of one or the channels 600 (shown in FIG. 7). In the example embodiment shown, each of the integral elongated protrusions 602C includes three fifth contacts 1210; however, other embodiments may be free of fifth contacts, include a single fifth contact, include two fifth contacts, or include more than fifth second contacts. Each of the fifth contacts 1210 may be electrically insulated from the other fifth contacts 1210, such as by plastic.

The scanner subassembly 120 may further include a scanner 1220. In at least one example embodiment, the scanner is barcode scanner (e.g., for reading one-dimensional and/or two-dimensional barcodes), an RFID reader, an NFC reader, a camera or any combination thereof. The scanner 1220 may be configured to scan, read, capture, and/or store an item, such as an item on a media bag. The item may include a one-dimensional barcode, a two-dimensional barcode, an RFID, an NFC tag, a number, an image or any combination thereof.

FIG. 13 is a schematic elevation view of a base of the tube joining device of FIG. 1 according to at least one example embodiment.

In at least one example embodiment, as shown in FIG. 13, the channels 600 of the base assembly 102 are uniformly spaced on each side 104B of the housing 104. For example, channels 600 on each side 104B of the housing 104 may be spaced apart by a first distance 1300. The uniform spacing facilitates modularity of the tube joining device 100 (shown in FIG. 1) such that the subassemblies (e.g., the scanner subassembly 120, the table subassembly 122, etc.) can be coupled to the base assembly 102 at any desired location. In at least one example embodiment, the tube joining device 100 may be configured to detect which, if any, subassembly is installed at each location, such as via data contacts.

In at least the example embodiment shown, each side wall 104B of the housing 104 includes two channels 600. In at least one other example embodiment, a housing includes one, three, four, five, or more channels, or is free of channels. In at least one other example embodiment, a housing includes elongated protrusions in addition to or as an alternative to channels. In at least one other example embodiment, additionally or alternatively, a housing includes channels and/or protrusions on a top wall and/or a bottom wall.

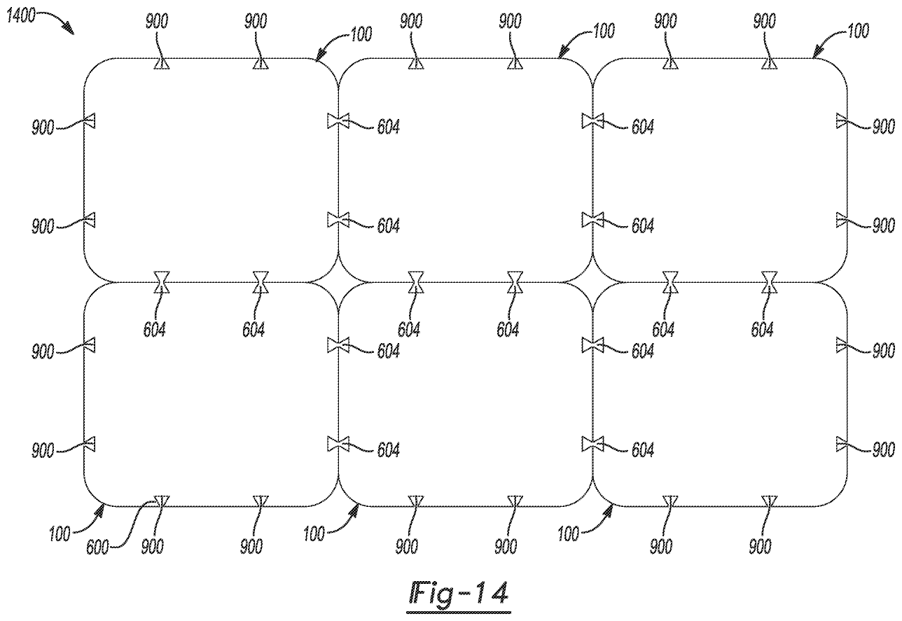

FIG. 14 is a schematic elevation view of a tube joining system including the tube joining device of FIG. 1 according to at least one example embodiment.

In at least one example embodiment, as shown in FIG. 14, multiple tube joining devices 100 may be coupled to one another to form a tube joining system 1400. The tube joining devices 100 may be configured to transfer power and data between one another via the contacts (see, e.g., first contacts 710, second contacts 810 in FIG. 11). Accordingly, cabling may be reduced or minimized compared to the same quantity of non-connected tube joining devices with distinct connections. Additionally, use of the tube joining system 1400 may facilitate increased speed compared to the same quantity of non-connected tube joining devices with distinct connections.

In at least one example embodiment, the respective base assemblies 102 may be connected via a plurality of the distinct elongated connectors 604. Channels 600 on a periphery of the system 1400 (i.e., not directly adjacent to another base assembly 102) may contain respective elongated blanks 900, as shown. Additionally or alternatively, channels 600 may be used to couple one or more subassemblies (e.g., the scanner subassembly 120, a power supply assembly (not shown), and the table subassembly 122) to one or more of the base assemblies 102.

At least one example embodiment relates to a tube joining device including elongated dovetail joints that form electrical, ground, and/or data connections. However, the elongated dovetail joints described herein may be applied to other devices to form connections with subassemblies, other devices, and/or structures.

Returning to FIG. 1, in at least one example embodiment, the tube joining device 100 is configured to execute one or more protocol assurance procedures. The protocol assurance procedures may reduce or prevent use of counterfeit cartridges (e.g., cartridge 112), used wafers (e.g., wafer 208), and/or defective wafers. Additionally or alternatively, the tube joining device 100 may be configured to ensure that various requirements are met as to the media to be combined. These procedures may improve efficiency and/or safety and/or reduce waste.

In at least one example embodiment, where requirements as to cartridge, wafer, and/or media bag are not met, the tube joining device 100 may be prevented from performing a joining procedure. Additionally or alternatively, the tube joining device 100 may be configured to generate an alert. The alert may include a visual display (e.g., a light and/or user interface device display), an audible alert, and/or a tactile alert. The alert may be at the tube joining device 100 and/or a remote device, such as a smart phone, a smart watch, or a remote computer.

The tube joining device 100 uses one or more consumables during operation, such as the cartridge 112 and/or wafers 208. In at least one example embodiment, a method of consumable authenticity verification is provided. The method may be performed by a device in which the consumable is intended to be used (e.g., the tube joining device 100) and/or at a separate authentication station. The method may be performed prior to, during, and/or after installation of the consumable in the device.

In at least one example embodiment, the method of consumable authenticity verification generally includes determining that a new consumable is, or should be, installed in a device; receiving data associated with the consumable; and determining whether the consumable is authentic (e.g., not counterfeit) based on the data. A determination that the consumable is not authentic may result in generation of an alert and/or preventing the device from performing a process using the consumable. At least one example embodiment includes a method of operation of the device including the method of consumable authenticity verification.

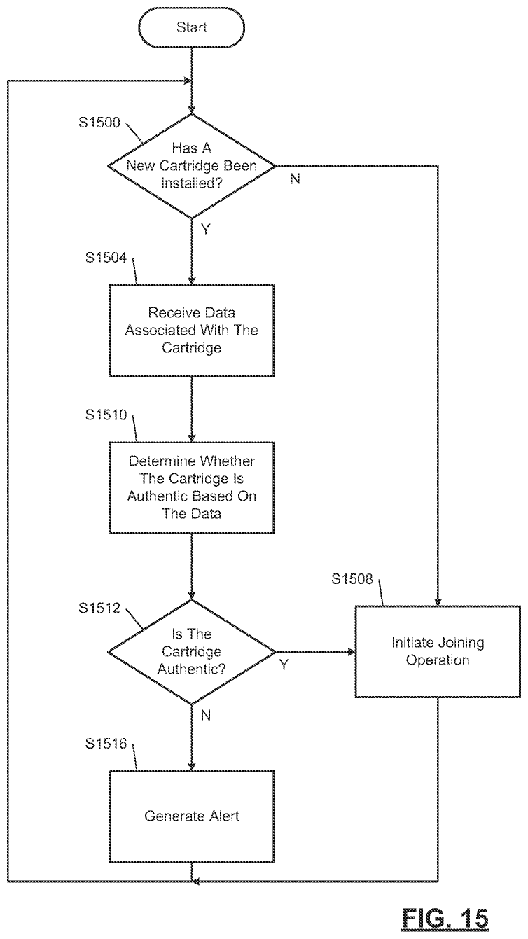

At least one example embodiment relates to the use of the cartridge 112 containing wafers 208 as a consumable in the tube joining device 100, as described in the discussion accompanying FIG. 15. However, the method of authenticity verification may be equally applied to other consumables and/or devices.

FIG. 15 is a flowchart depicting a method tube joining including cartridge authenticity verification according to at least one example embodiment.

In at least one example embodiment, as shown in FIG. 15, a method of tube joining including cartridge authenticity verification is provided. In at least one example embodiment, the method is performed using the tube joining device 100 including the scanner 402; however, the method may be performed using other tube joining devices according to example embodiments of the present disclosure. Additionally or alternatively, the method may be performed at a separate authentication station. The cartridge verification may be performed periodically at predetermined (or alternatively, desired) time intervals, at the time of known or expected cartridge change, prior to each weld, on demand (e.g., based on operator decision), and/or randomly during use, for example. The method begins at S1500.

At S1500, a control module determines whether a new cartridge has been installed. In at least one example embodiment, the control module determines that a new cartridge has been installed based on output from a sensor (e.g., an optical sensor) and/or a process counter associated with level of use (e.g., weld count). In other example embodiments, a control module may determine whether a new consumable has, or should be, installed based on device uptime, passage of time (e.g., after a predetermined or desired time has passed since installation of the consumable), a measurement indicative of wear, and/or operator input (e.g., indicating a decline in device output quality and/or device performance).

In at least one example embodiment, the control module determines that a new cartridge has been installed based on output from a sensor. For example, the sensor may be coupled to a component, such as a latch, that is actuated each time a cartridge is removed or inserted. In at least one example embodiment, the sensor includes an optical sensor. In at least one other embodiment, the control module determines that a new cartridge has been installed based on output from a limit switch.

In at least one other example embodiment, the control module determines that a new cartridge has been installed based on weld count. In example embodiments, weld count may be associated with the quantity of wafers used and/or removed from the cartridge. In at least one example embodiment, weld count is based on data collected regarding proper tube placement, heating of the wafer 208, cutting movement of the wafer 208, alignment movement of the tubing clamp 106, and/or resetting of the tubing clamp 106 to a home position.

The control module may determine that a new cartridge has been installed after the tube joining device has made a predetermined (or alternatively, desired) quantity of welds. In at least one example embodiment, the predetermined quantity of welds is greater than or equal to about 5 welds (e.g., greater than or equal to about 10 welds, greater than or equal to about 20 welds, greater than or equal to about 30 welds, greater than or equal to about 40 welds, greater than or equal to about 50 welds, or greater than or equal to about 60 welds). The predetermined quantity of welds may be less or equal to about 200 welds (e.g., less than or equal to about 175 welds, less than or equal to about 150 welds, less than or equal to about 125 welds, less than or equal to about 100 welds, less than or equal to about 90 welds, or less than or equal to about 80 welds).

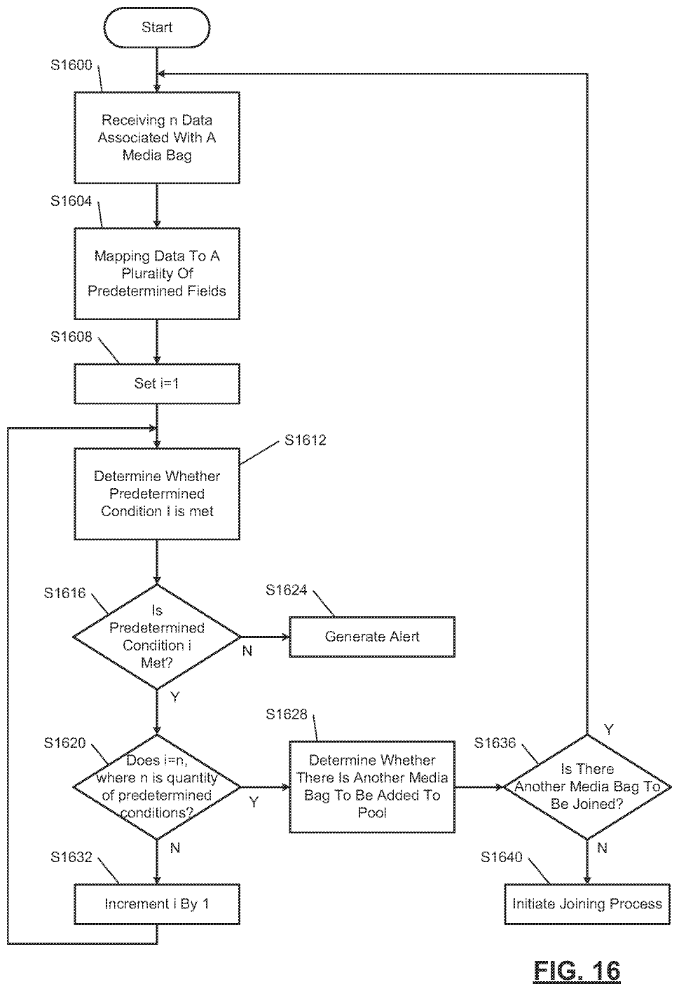

If a new cartridge has been installed, the method proceeds to S1504. Otherwise, the method proceeds to S1508. At S1508, the control module permits and/or initiates a joining operation. In at least one example embodiment, initiating a joining operation may include the method of FIGS. 16 and/or 18, described in greater detail below. In at least one other example embodiment, if a new cartridge has not been installed, control takes no action and operation of the tube joining device may proceed.

At S1504, the control module receives data associated with the cartridge. Receiving data associated with the cartridge may include scanning, with a scanner, an item on the cartridge. The item may include a one-dimensional barcode, a two-dimensional barcode, an RFID tag, an NFC tag, a number, a series of letters, an alpha-numeric code, an image, or any combination thereof. The scanner may include a barcode scanner, an RFID reader, an NFC reader, a camera, or any combination thereof. Prior to receiving the data, the method may include controlling the scanner to detect the data (e.g., scan the item). The method continues at S1510.

At S1510, the control module determines whether the cartridge is authentic based on the data. In at least one example embodiment, the control module determines whether the cartridge is authentic based on a “white list” method. The white list method includes comparing the data to a predetermined list of data to determine whether the data is present on the list. If the data is on the list, then the cartridge is authentic. Otherwise, the cartridge is assumed to be a counterfeit. The method may include controlling the scanner to detect the data (e.g., scan the item), comparing the data to a list of data stored in memory, determining that the cartridge is authentic if the data is on the list, and/or determining that the cartridge is not authentic if the data is not on the list.

In at least one other example embodiment, the control module determines whether the cartridge is authentic based on a “solve cypher” method. In the solve cypher method, the barcode includes information to facilitate solving of a cypher. If the cypher can be solved, the cartridge is identified as authentic. Otherwise, the cartridge is assumed to be a counterfeit. The method may include controlling the scanner to detect the data (e.g., scan the item), solving the cipher based on the data and/or information stored in memory, determining that the cartridge is authentic if the cypher is solved, and/or determining that the cypher is not authentic if the cypher cannot be solved. The method continues at S1512. At S1512, if the cartridge is authentic, the method proceeds at 1508. Otherwise, the method continues at S1516.

At S1516, the control module generates an alert and/or alarm. The alert may be visual, audible, and/or tactile, as described above. The method returns to S1500. In at least one other example embodiment, if control determines that the cartridge is not authentic, control prevents the tube joining device from performing a joining operation.

In at least one example embodiment, when the item is an RFID tag, the method further includes writing information to the RFID tag. The information may include count of welds made from wafers in the cartridge, authenticity status, empty status, wafers remaining in cartridge, dates and/or times cartridge was installed in device and/or wafers were used, and/or other data associated with use (e.g., information associated with media bags connected to tubes joined using wafers in cartridge). The writing may be performed at any point in the method, including prior to or after a joining operation at S1508 and/or after a determination on cartridge authenticity at S1512.

In at least one example embodiment, when first cartridge is replaced with a second cartridge, one or more wafers (e.g., two wafers) from the first cartridge remain in the tube welding device after the cartridge is changed. Accordingly, the method may include different or additional steps to account for the wafers from the first cartridge. For example, upon a determination that the second cartridge has been installed, the tube welding device may reject a predetermined (or alternatively, desired) quantity of wafers (i.e., the remaining wafers from the first cartridge) prior to performing a joining operation.

FIG. 16 is a flowchart depicting a method of verifying media combination protocol according to at least one example embodiment.

In at least one example embodiment, as shown in FIG. 16, a method of verifying media combination protocol is provided. In at least one example embodiment, the method is performed using the tube joining device 100 including the scanner subassembly 120; however, the method may be performed using other tube joining devices according to example embodiments of the present disclosure.

In at least one example embodiment, the contents of multiple media source bags (also referred to as “parent bags” or “mother bags”) are combined into a single pool bag (also referred to as a “daughter bag”). In at least one example embodiment, such as when an overhead scanner is used (e.g., the scanner subassembly 120) each source bag may be scanned, verified, and then added to the pool bag prior to scanning another source bag. In at least one other example embodiment, all source bags may be scanned (e.g., with a handheld barcode scanner) and verified prior to combining the contents of any source bag into the pool bag.

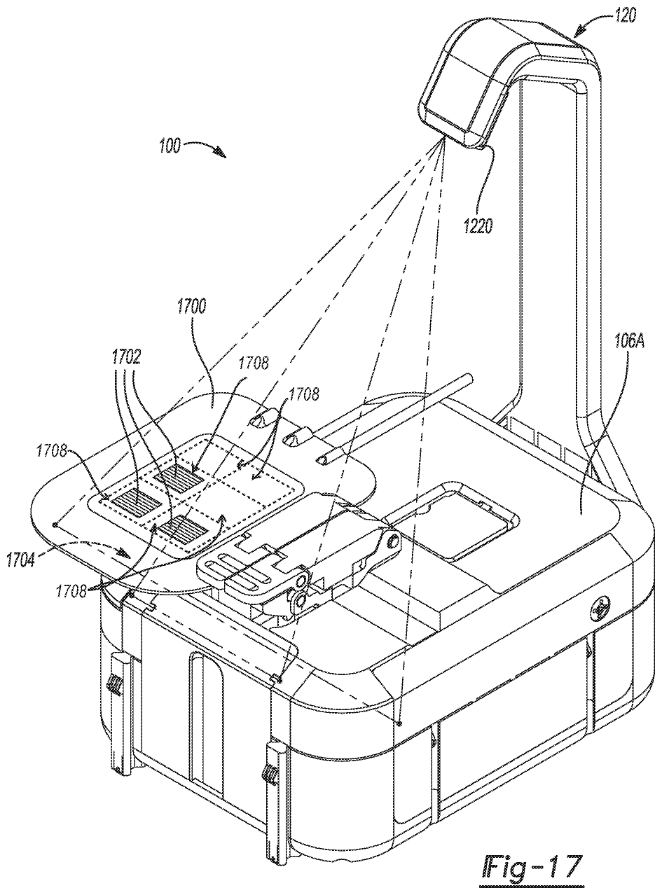

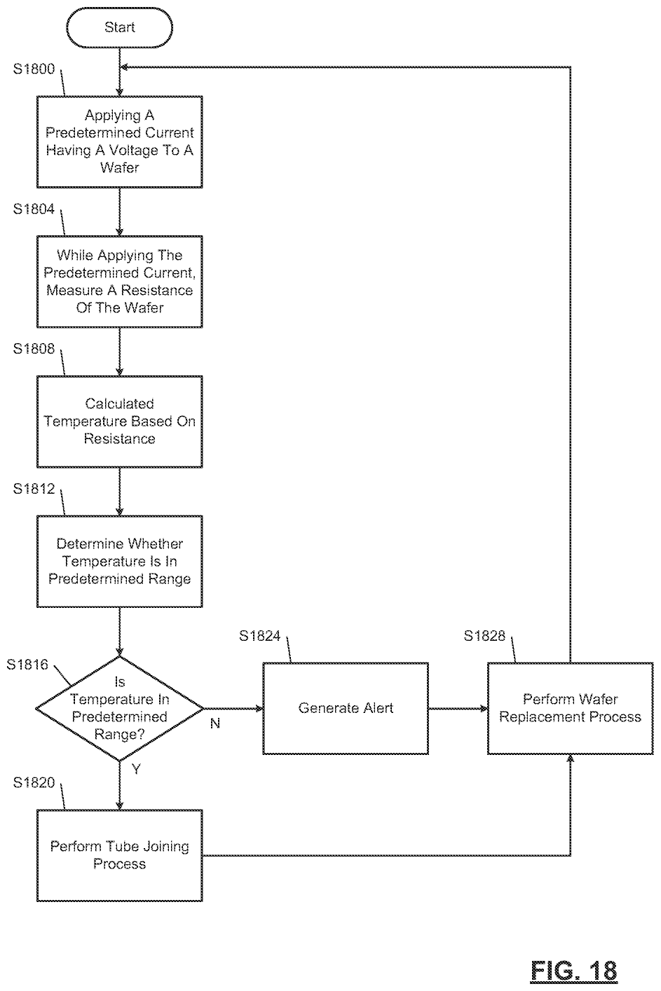

FIG. 17 is a perspective view of the tube joining device of FIG. 1 having a media bag on a top wall according to at least one example embodiment.

In at least one example embodiment, as shown in FIG. 17, the tube joining device 100 may be used to combine the contents of a plurality of media bags 1700. The plurality of media bags 1700 may be combined to form a pool of media. Each of the media bags 1700 may include one or more items, such as a plurality of items 1702 thereon. The items 1702 may be or include one-dimensional barcodes, two-dimensional barcodes, RFIDs, NFC tags, numbers, a series of letters, an alpha-numeric code, and/or an image. The scanner 1220 of the scanner subassembly 120 may be configured to read and/or scan the plurality of items 1702, concurrently. The scanner 1220 may configured to scan or read within a predetermined region, such as all or a portion (e.g., a scan region 1704) of the top wall 104A and/or all or a portion of a surface of the media bag. In at least one example embodiment, each of the items 1702 may be associated with the scan region 1704 or one or more subregions 1708. In at least one example embodiment, each of the items 1702 is associated with exactly one of the respective subregions 1708. In at least one other example embodiment, the scanner 1220 may be configured to scan or read the plurality of items 1702 sequentially, such as buy sequentially scanning within a plurality of predetermined (or alternatively, desired) regions.

Returning to FIG. 16, the method begins at S1600. At S1600, a control module receives data items associated with a media bag. The control module may receive the data items by scanning, with a scanner, the items on the media bag. The method may include controlling the controller to scan or read the data items. In at least one example embodiment, the scanner scans all the items concurrently. In at least one other example embodiment, the scanner scans the items sequentially. In at least one example embodiment, the data items include barcodes. In at least one example embodiment, the data items include an RFID containing data. The method continues at S1604.

At S1604, the control module maps the data into a plurality of predetermined fields. In at least one example embodiment, the control module maps n data into n predetermined fields. The control module may map the data into the predetermined fields based on a unique identifier associated with the respective data. In at least one example embodiment, the predetermined fields include bag identification; unit identification (an identification that is unique to a particular media bag); machine identification (e.g., identification of a machine used to collect the media); storage solution for media contained in media bag; processing step; and/or, when the media is blood components, blood type; viral status (e.g., cytomegalovirus) of the blood; whether the blood has undergone testing; and/or sex of donor. The method may further include storing the mapped data in a memory of the tube joining device. The method continues at S1608. At S1608, the control module sets i=1. The method continues at S1612.

At S1612, the control module determines whether the mapped data meets a predetermined condition. The determination may be based on data stored in the memory of the tube joining device, the mapped data, and/or pool data. The pool data may be related to other media bags for the pool that have already been scanned. The pool data may be stored in the memory of the tube joining device. In at least one example embodiment, data may fail to meet a predetermined condition when the media bag includes an incorrect and/or unexpected blood type; the addition of the media to the pool would result in the pool include blood from two or more female donors; the addition of the media to the pool would result in too many units of blood in the pool; and/or incorrect and/or unexpected processing step, etc. The method continues at S1616.

At S1616, the method moves to S1620 if the predetermined condition is met. Otherwise, if the predetermined condition is not met, the method proceeds at S1624.

At S1620, the control module determines whether i=n. When i=n, all of the predetermined conditions have been analyzed. If i=n, the method proceeds at S1628. Otherwise, if ien, then there are additional predetermined conditions to analyze and the method proceeds to S1632.

At S1632, the control module increments i by 1. The method returns to S1612.

At S1624, the control module generates an alert. The alert may be visual, audible, and/or tactile, as described above. In at least one example embodiment, additionally or alternatively, the control module prevent a joining operation until a different media bag is scanned and verified.

At S1628, the control module determines whether there is another media bag to be added to the pool. In at least one example embodiment, the determination may be based on an expected quantity of media bags in the pool and/or units of blood. Expected quantity or units may be based on data input by the operator and/or stored in the memory. In at least one example embodiment, the user may indicate, such as by interacting with a user interface device, that there is another media bag to be added to the pool. The method proceeds to S1636.

At S1636, the control module returns to S1600 if there is another media bag to be scanned. Otherwise, the method continues to S1640.

At S1640, the control module initiates a joining process. Initiating a joining process may include performing the method of FIG. 18. In at least one other example embodiment, the control module does not prevent a joining process.

In at least one example embodiment, the method may further include writing to an RFID tag on the media bag. In at least one example embodiment, the method includes writing information about media bag contents to an RFID tag on a pool bag. The information may further include dates of collection and/or combination of contents, identification of machines used, etc. Any of the information contained on source bags may be written to the pool bag. This step may be performed at any desired point after S1600 in the method.

FIG. 18 is a flowchart depicting a method of performing a tube joining process with wafer verification according to at least one example embodiment.

In at least one example embodiment, as shown in FIG. 18, a method of performing a tube joining process with wafer verification is provided. In at least one example embodiment, the method is performed using the tube joining device 100 including the tube joining subassembly 105; however, the method may be performed using other tube joining devices according to example embodiments of the present disclosure. The method begins at S1800.

At S1800, a control module applies a predetermined (or alternatively, desired) current to a wafer. The current may be applied at the electrical contacts 500, as shown in FIG. 5. The current may flow through the resistive trace 304 to heat the body 300 of the wafer 208 (shown in FIG. 3). The method continues at S1804.

At S1804, while applying the predetermined current, the control module measures a resistance of the wafer. The control module may measure the resistance, such as by using an ohm meter. The resistance may be measured at the contacts 500 (shown in FIG. 5). The method continues at S1808.

At S1808, the control module calculates a temperature associated with the resistance. The control module may calculate the temperature based on the resistance and a known relationship between resistance and temperature stored in memory. The method continues at S1812.

At S1812, the control module determines whether the temperature is within a predetermined (or alternatively, desired) temperature range. In at least one example embodiment, temperatures outside of (e.g., above) the predetermined temperature range may indicate the wafer is used and/or defective. The control module may determine that the temperature is outside of the predetermined temperature range by comparing the temperature with the predetermined range stored on the memory. The method continues at S1816.

At S1816, if the temperature is in the predetermined range, then the method continues to S1820. Otherwise, if the temperature is not in the predetermined range, then the method continues at S1824. In at least one other example embodiment, the control module performs a predetermined (or alternatively, desired) quantity of repetitions of S1800, S1804, S1808, S1812, and S1816. The method may require a predetermined (or alternatively, desired) quantity of positive verifications at S1816 before proceeding to S1820 and/or require a predetermined (or alternatively, desired) quantity of negative determinations at S1816 before proceeding to S1824.

In at least one example embodiment, At S1820, the control module performs a tube joining process. The method continues at S1828. At S1828, the control module performs a wafer replacement process to move a wafer from the cartridge to the pre-joining position shown in FIG. 5. In at least one example embodiment, the method further includes periodically include changing a cartridge, such as when there are no unused wafers remaining in the cartridge. The method returns to S1800.

At S1824, the control module generates an alert. The alert may be visual, audible, and/or tactile, as described above. Additionally or alternatively, the control module may prevent a tube joining operation. The method continues to S1828.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method of initiating a tube joining process comprising:
receiving, with a tube joining device, data associated with a cartridge;
determining whether the cartridge is authentic based on the data; and
in response to a determination that the cartridge is authentic, initiating the tube joining process.

2. The method of claim 1, wherein the determining includes
comparing the data to a predetermined list of data, and
determining that the cartridge is authentic when the data is included in the predetermined list of data.

3. The method of claim 1, wherein the receiving includes scanning, with a scanner, an item.

4. The method of claim 3, wherein the item is a one-dimensional barcode, a two-dimensional barcode, a radiofrequency identification, a near-field communication tag, or any combination thereof.

5. The method of claim 1, further comprising:
in response to a determination that the cartridge is not authentic, generating an alert.

6. A method of verifying media combination protocol comprising:
verifying a first media bag including,
receiving, with a tube joining device, first data associated with the first media bag, the receiving including concurrently scanning, with a scanner, a first plurality of items;
mapping the first plurality of items to a first plurality of predetermined fields; and
after the mapping, determining whether a first predetermined condition is met.

7. The method of claim 6, wherein the determining includes determining whether a respective first predetermined condition is met for each of the first plurality of predetermined fields.

8. The method of claim 6, further comprising:
verifying a second media bag including,
receiving, with the tube joining device, second data associated with the second media bag, the receiving including concurrently scanning, with a scanner, a second plurality of items;
mapping the second plurality of items to a second plurality of predetermined fields; and
after the mapping, determining whether a second predetermined condition is met.

9. The method of claim 8, wherein the determining whether the first predetermined condition is met is based on the first data and data stored in a memory of the tube joining device.

10. The method of claim 9, wherein the determining whether the second predetermined condition is met is based on the first data, the second data, and the data stored in the memory of the tube joining device.

11. The method of claim 8, further comprising:
performing, with the tube joining device, a tube joining process based on a determination that the first predetermined condition and the second predetermined condition are met.

12. The method of claim 6, further comprising:
storing, in a memory of the tube joining device, at least a portion of the first data associated with the first media bag.

13. The method of claim 6, wherein the first plurality of items includes a plurality of one-dimensional barcodes, a plurality of two-dimensional barcodes, a plurality of radiofrequency identifications, or a plurality of near-field communication tags.

14. The method of claim 6, wherein the determining is based on the first plurality of items and data stored in a memory of the tube joining device.

15. The method of claim 6, further comprising:
based on a determination that the first predetermined condition is not met, generating an alert.

16. The method of claim 6, wherein the first data associated with the first media bag includes media bag identification, storage solution, sex of donor, viral status of media, blood type, quantity of media units, unit identification, process step, or any combination thereof.

17. A method of performing a tube joining process comprising:
applying a current to a wafer;
determining a temperature of the wafer during the applying;
determining whether the temperature is within a predetermined temperature range; and
based on a determination that the temperature is within the predetermined temperature range, performing, with a tube joining device, a tube joining process.

18. The method of claim 17, wherein the determining the temperature includes,
detecting an electrical resistance of the wafer, and
calculating the temperature based on the electrical resistance.

19. The method of claim 17, further comprising:
based on a determination that the temperature is outside of the predetermined temperature range, generating an alert.

20. The method of claim 17, further comprising:
based on a determination that the temperature is outside of the predetermined temperature range, replacing the wafer.
